(19) United States
(12) Patent Application Publication
(10) Pub. No.: US 2024/0157102 A1
(43) Pub. Date: May 16, 2024

(54) INTEGUMENTAL DISSOLVING NEEDLES AND NEEDLE DEVICES

(71) Applicant: Norihiro NANGOU, Tokyo (JP)

(72) Inventor: Norihiro NANGOU, Tokyo (JP)

(21) Appl. No.: 18/416,881

(22) Filed: Jan. 18, 2024

Related U.S. Application Data
Application No. 16/714,821 is a continuation of application No. PCT/JP2018/010912, filed on Mar. 19, 2018.
Application No. PCT/JP2018/010912 is a continuation of application No. 16/714,821, filed on Dec. 16, 2019.

(30) Foreign Application Priority Data
Jun. 22, 2017 (JP) 2017-121980

(51) Int. Cl. A61M 37/00; A01G 7/06; A01N 25/12

(52) U.S. Cl. CPC A61M 37/00; A01G 7/06; A01N 25/12; A61M 2205/0238

(57) ABSTRACT

There currently exists no needle capable of delivering pharmaceutical or cosmetic ingredient(s) into deep layers of integumental tissue (e.g. skin, scales, bark), nor any needle device designed to facilitate the precise administration of a desired dose, and to limit inflammation, pain, and other side effects associated with the needle(s). The present invention relates to an integumental dissolving needle—which houses micronized cosmetic/pharmaceutical ingredient(s), encapsulated by a coating agent absorbed into the integument—arranged on the surface or surfaces of a poultice. Needle thickness and length may be varied according to the biological species of interest. In addition, dosage(s) is/are printed on each section of the device to make explicit how many milligrams of the ingredient are present per unit area; these sections may be separated from one another along groove(s) or other markers on the device. The present invention is a needle device consisting of the above components.

### CROSS-REFERENCE TO RELATED APPLICATIONS

This application is a continuation of U.S. patent application Ser. No. 16/714,821, filed on Dec. 16, 2019, which is a continuation of International Patent Application No. PCT/JP2018/010912, filed on Mar. 19, 2018, which claims priority to Japanese Patent Application No. 2017-121980, filed in JP on Jun. 22, 2017, the entire of the contents of each of which are incorporated herein by reference BACKGROUND

### 1. TECHNICAL FIELD

The present invention relates to integumental dissolving needles capable of delivering pharmaceutical or cosmetic ingredients into deep layers of integumental tissue (e.g. skin, scales, bark); and needle devices incorporating them designed to facilitate the precise administration of a desired dose, and to limit inflammation, pain, and other side effects associated with their application.

### 2. RELATED ART

Conventional intradermal dissolving microneedles (“MNs”) such as those described in Patent Refs. 1 and 2 can deliver pharmaceutical or cosmetic ingredients into the upper layers of human skin (e.g. epidermis, stratum corneum), but are unable to reach the deepest layers of human skin. While large MN arrays employing a needle length of 800 m, such as that described in Non-Patent Ref. 1, can deliver pharmaceutical or cosmetic ingredients as deep as the human dermis, they cause pain in the skin after their application. Human skin ranges from 1-4 mm in thickness (Non-Patent Ref 2); however, cow skin is 5-7 mm in thickness, and dog skin is exceedingly thin (Non-Patent Ref. 3). This variability requires users to select MN devices having a needle length suitable for the species of interest. Moreover, conventional MN arrays do not intuitively indicate how many milligrams of ingredient(s) are present in a given unit area, nor do they employ grooves or perforations to facilitate the sectioning of the array, nor are such arrays ‘pre-sectioned’ for sale. The absence of such elements makes it difficult to precisely administer a desired dose.

### RELATED ART DOCUMENTS

### Patent Literature

[Patent Reference 1] Published unexamined patent application 2010-82401. In Japanese.
[Patent Reference 2] Published unexamined patent application 2012-25723. In Japanese.

### Non Patent Literature

[Non-Patent Reference 1] Advanced Science, Technology & Management Research Institute of Kyoto. [2011 Strategic Foundational Technology Improvement Support Operation, R&D Report: Development of novel tip-loaded drug-delivery microneedles, and applications to hair growth formulations.] March 2012. In Japanese. http://www.chusho.meti.go.jp/keiei/sapoin/portal/seika/2010/22h-73.pdf
[Non-Patent Reference 2] Hisashi Ishihara. [The Structure of the Skin.] 10 Apr. 2015. In Japanese. http://www.ams.eng.osaka-u.ac.jp/user/ishihara/?p=432
[Non-Patent Reference 3]: Kaneko Mikihiro. [5. Learn How the Body Works, 30: Learning How the Skin Works (to Raise a Healthy Horse).] In Japanese. http://www.b-t-c.or.jp/btc_p300/btcn/btcn68/btcn068-04.pdf

### GENERAL DISCLOSURE

The present invention was developed to solve the following problems:
How to facilitate the delivery of a pharmaceutical or cosmetic ingredient of interest into the deepest layers of integumental tissue (e.g. skin, scales, bark);
How to limit subsequent inflammation, pain, and other side effects associated with needle device(s); and
How to facilitate the precise administration of a desired amount of ingredient(s) by indicating dosage or dosages in an intuitive way, i.e. how many milligrams of the ingredient(s) are present in a given unit area.

The present invention provides an integumental (e.g. skin, scales, bark) dissolving needle, which is filled with micronized pharmaceutical ingredient(s) or micronized cosmetic ingredient(s) encapsulated by a layer of coating agent (“coating layer”) that is absorbed into the integument (e.g. skin, scales, bark), to allow the said ingredient(s) to penetrate into deep layers of the integument (e.g. skin, scales, bark). Needle thickness and length may be varied according to the biological species of interest.

The present invention provides an integumental (e.g. skin, scales, bark) dissolving needle fabricated such that suitable pharmaceutical ingredient(s) or cosmetic ingredient(s) are an integral component of the needle itself, to directly administer the said ingredient(s) without needing to wait for the needle to dissolve. Needle thickness and length may be varied according to the biological species of interest.

The present invention provides an integumental (e.g. skin, scales, bark) dissolving needle fabricated such that suitable pharmaceutical ingredient(s) covered with a coating layer, or cosmetic ingredient(s) covered with a coating layer, are an integral component of the needle itself, to administer the said ingredient(s) (without needing to wait for the needle to dissolve) and to simplify the manufacturing process. Needle thickness and length may be varied according to the biological species of interest.

The present invention provides an integumental (e.g. skin, scales, bark) dissolving needle device, in which needle(s) are arranged on the application-side surface of a poultice or surfaces of a poultice (e.g. hot compress, cold compress, anti-inflammatory analgesic tape), to limit subsequent inflammation, pain, and other side effects associated with needle(s).

The present invention provides an integumental dissolving needle device, on which dosage or dosages is printed to clearly indicate how many milligrams of ingredient(s) are present in a given unit area, and which contains groove(s) or perforations in the said device to facilitate the precise administration of a desired dose. For example, an embodiment might facilitate the removal of 1 cm² unit(s), each containing 10 mg (or 1.25 mg, etc.) of a pharmaceutical ingredient, by sectioning the needle device into 1 cm² units by groove(s) or perforations, and having “10 mg” (“1.25 mg”, etc.) printed on each unit. These characteristics make it easier for a user to break, cut, or otherwise divide the device and administer the desired dose.

The present invention provides an integumental dissolving needle device, on which dosage or dosages is printed to clearly indicate how many milligrams of ingredient(s) are present in a given unit area, and which is pre-sectioned to facilitate the precise administration of a desired dose. For example, a possible embodiment is pre-sectioned, 1 cm² units, each containing 10 mg (or 1.25 mg) of a pharmaceutical ingredient, and on each of which is printed “10 mg” (or “1.25 mg”, etc.). One or more units could then be applied to administer the desired dose.

The integumental (e.g. skin, scales, bark) dissolving needle, filled with micronized pharmaceutical or cosmetic ingredient particle(s) encapsulated by a coating agent that is absorbed into the integument (e.g. skin, scales, bark), offers the beneficial effects of allowing the encapsulated granules to penetrate deep into the integument (e.g. skin, scales, bark) once the needle itself dissolves in the integument (e.g. skin, scales, bark). This design offers superior penetrability to conventional MN(s).

The integumental dissolving needle fabricated such that suitable pharmaceutical ingredient(s) or cosmetic ingredient(s) are an integral component of the needle itself, offers the beneficial effects of allowing the said ingredient(s) to be administered directly, without waiting for the needle to dissolve, thereby allowing the said ingredient(s) to quickly penetrate into the integument.

The integumental dissolving needle fabricated such that suitable pharmaceutical ingredient(s) encapsulated by a coating layer, or suitable cosmetic ingredient(s) encapsulated by a coating layer, are an integral component of the needle itself, offers the beneficial effects of allowing the said ingredient(s) to be administered (without needing to wait for the needle to dissolve), and to simplify the manufacturing process, thereby allowing the said ingredient(s) to quickly penetrate into the integument.

The integumental (e.g. skin, scales, bark) dissolving needle device, in which any of the needles described above are arranged on the application-side surface of a poultice or surfaces of a poultice (e.g. hot compress, cold compress, anti-inflammatory analgesic tape), offers the beneficial effects of minimizing subsequent inflammation, pain, and other side effects associated with the needle(s).

The integumental dissolving needle device, on which dosage or dosages is printed and which contains groove(s) or perforations, offers the beneficial effect of facilitating the precise administration of a desired dose. For example, an embodiment might facilitate the removal of 1 cm² unit(s), each containing 10 mg (or 1.25 mg, etc.) of a pharmaceutical ingredient, by sectioning the needle device into 1 cm² units by groove(s) or perforations, and having “10 mg” (“1.25 mg”, etc.) printed on each unit. These characteristics make it easier for a user to break, cut, or otherwise divide the device and administer the desired dose.

The integumental dissolving needle device, on which dosage or dosages is printed and which is pre-sectioned, offers the beneficial effect of facilitating the selection of a desired dose. For example, a possible embodiment has pre-sectioned, 1 cm² units, each containing 10 mg (or 1.25 mg) of a pharmaceutical ingredient, and each is printed with “10 mg” (“1.25 mg”, etc.). One or more units could then be applied to administer the desired dose.

### BRIEF DESCRIPTION OF THE DRAWINGS

FIG. 1. An integumental dissolving needle device with dosage printed on surface (groove type; perspective view)

FIG. 2. An integumental dissolving needle device with dosage printed on surface (perforation type; perspective view)

FIG. 3. An integumental dissolving needle device with dosage printed on surface (pre-sectioned type; perspective view)

FIG. 4. An integumental dissolving needle device housing cosmetic or pharmaceutical ingredient(s), that is a nanoparticle or that is a micronized particle having a size smaller than a nanometer order, covered with a layer of coating agent that is absorbed into the integument (cross-section view).

FIG. 5. An integumental dissolving needle device housing granules of different layer structures. In principle, granules may have any plural number of layers. The figure depicts a specific embodiment containing: two-layer granules, consisting of cosmetic or pharmaceutical ingredient(s), that is a nanoparticle or that is a micronized particle having a size smaller than a nanometer order, encapsulated by a layer of coating agent that is absorbed into the integument; as well as four-layer granules, consisting of the said (two-layer) granules further covered with a layer of micronized cosmetic or pharmaceutical ingredient(s), followed by another layer of coating agent (cross-section view).

FIG. 6. An integumental dissolving needle device housing multi-layer granules consisting of cosmetic or pharmaceutical ingredient(s) that is a nanoparticle or that is a micronized particle having a size smaller than a nanometer order, which are encapsulated by a layer of coating agent that is absorbed into the integument, which is further covered with a layer of micronized cosmetic or pharmaceutical ingredient(s), followed by another layer of coating agent (cross-section view).

### DESCRIPTION OF EXEMPLARY EMBODIMENTS

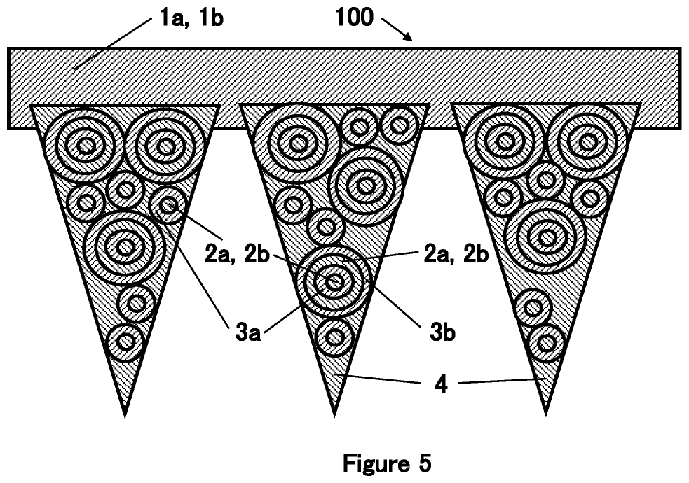

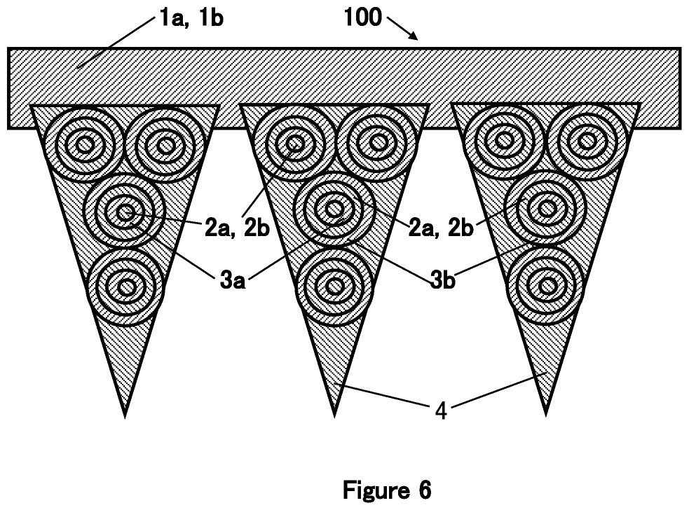

In one embodiment, a cosmetic ingredient 2b that is a nanoparticle or that is a micronized particle having a size smaller than a nanometer order (or similarly micronized pharmaceutical ingredient particle 2a) is encapsulated by a layer of coating agent 3a that is absorbed into the integument (e.g. skin, scales, bark). These granules are housed in an integumental (e.g. skin, scales, bark) dissolving needle 4. Granules may possess more than two layers: FIGS. 5 and 6 depict four-layer granules, composed of the two-layer granules described immediately above, further coated with an additional layer of micronized cosmetic ingredient 2b (or pharmaceutical ingredient 2a), followed by an additional layer of coating agent 3b. Some such capsules depicted in FIG. 5, and all depicted in FIG. 6, have a four-layer structure; however, granules of more than four layers are possible. Integumental dissolving needle 4 thickness and length may be varied according to the biological species of interest.

If possible, the integumental dissolving needle 4 may be fabricated such that the pharmaceutical ingredient 2a (or cosmetic ingredient 2b, or coated pharmaceutical ingredient 2a, or coated cosmetic ingredient 2b) is an integral component of the integumental dissolving needle 4 itself. In this case, the coated pharmaceutical ingredient 2a or cosmetic ingredient 2b merely housed in the integumental dissolving needle 4 may differ from the cosmetic ingredient 2b (or pharmaceutical ingredient 2a, or coated pharmaceutical ingredient 2a, or coated cosmetic ingredient 2b) present in the integumental dissolving needle's 4 composition. For example, an integumental dissolving needle 4 might house an encapsulated hypertension drug as the pharmaceutical ingredient 2a, while compositionally containing an antibacterial agent. Integumental dissolving needle 4 thickness and length may be varied according to the biological species of interest.

In another embodiment, the integumental dissolving needle(s) 4 described above may be arranged on the application-side surface of a poultice 1a or surfaces of a poultice 1a (e.g. hot compress, cold compress, anti-inflammatory analgesic tape). Alternatively, the integumental dissolving needle(s) 4 may be arranged on a patch 1b if anti-inflammatory drug-containing poultices 1a cannot be used (e.g. if the integumental dissolving needle device 100 is for use by a person (or species) allergic to an anti-inflammatory drug or analgesic, or a person (or species) that does not respond to the anti-inflammatory drug or analgesic).

The composition of the coating agents 3a, 3b shall include at least one of the following biocompatible substances: nucleic acid esters, nucleotides, cholesterol, sucrose fatty acid esters, fatty acid esters such as glycerin fatty acid esters, fatty acids containing cutin, fatty acid salts, phosphate, phosphate esters containing phospholipids, polylactic acid salts, polylactic acid esters (including polylactic acid and polyglycolic acid copolymers), saccharides (including mucopolysaccharides [e.g. hyaluronic acid], dextran, maltose, glucose, sucrose, galactose, lactose, cellulose, carboxymethyl cellulose, hydroxyethyl cellulose, trehalose, peptidoglycans, polyglycolic acid, and chitin), amino acid esters, amino acid salts, proteins (e.g. gelatin, collagen, keratin), biodegradable polymers (e.g. peptides, lignin, polyvinyl alcohol, polyvinyl pyrrolidone), fullerene, vitamins, hormones, antigens, antibodies, substrates, and enzymes; alternatively or additionally, derivatives of any of these substances, and/or some mixture of them. The substances above are given as examples: any biocompatible substance capable of encapsulating the cosmetic ingredient 2b or pharmaceutical ingredient 2a, and being absorbed into the integument (e.g. skin, scales, bark) of the species of interest, may be used as (or in) the coating agents 3a, 3b.

The composition of the aforementioned integumental dissolving needle 4 shall include at least one of the following biocompatible substances: nucleic acid esters, nucleotides, cholesterol, sucrose fatty acid esters, fatty acid esters including glycerin fatty acid esters, fatty acids containing cutin, fatty acid salts, phosphate, phosphate esters containing phospholipids, polylactic acid salts, polylactic acid esters (including polylactic acid and polyglycolic acid copolymers), saccharides (including mucopolysaccharides [e.g. hyaluronic acid], dextran, maltose, glucose, sucrose, galactose, lactose, cellulose, carboxymethyl cellulose, hydroxyethyl cellulose, trehalose, peptidoglycans, polyglycolic acid, and chitin), amino acid esters, amino acid salts, proteins (e.g. gelatin, collagen, keratin), biodegradable polymers (e.g. peptides, lignin, polyvinyl alcohol, polyvinyl pyrrolidone), fullerene, vitamins, hormones, antigens, antibodies, substrates, and enzymes; alternatively or additionally, derivatives of any of these substances, and/or some mixture of them. The substances above are given as examples: any biocompatible substance capable of composing the integumental dissolving needle 4, and being absorbed into the integument (e.g. skin, scales, bark) of the species of interest, may be used as (or in) the integumental dissolving needle.

Additionally, the present invention may be embodied in an integumental dissolving needle device 100, on which dosage or dosages is printed to clearly indicate how many milligrams of ingredient(s) are present in a given unit area; and which contains grooves or perforations to facilitate the separation of units, or which is pre-sectioned into the corresponding units. Groove(s) 10 may be located on the same side of the integumental dissolving needle device 100 as the integumental dissolving needles 4, the opposite side, or both sides. For example, such an embodiment might facilitate the removal of 1 cm² unit(s), each containing 10 mg (or 1.25 mg, etc.) of a pharmaceutical ingredient 2a by sectioning the integumental dissolving needle device 100 into 1 cm² units by grooves or perforations, or physically pre-sectioning the integumental dissolving needle device 100 into similar unit(s), and having “10 mg” (“1.25 mg”, etc.) printed on each unit. Examples

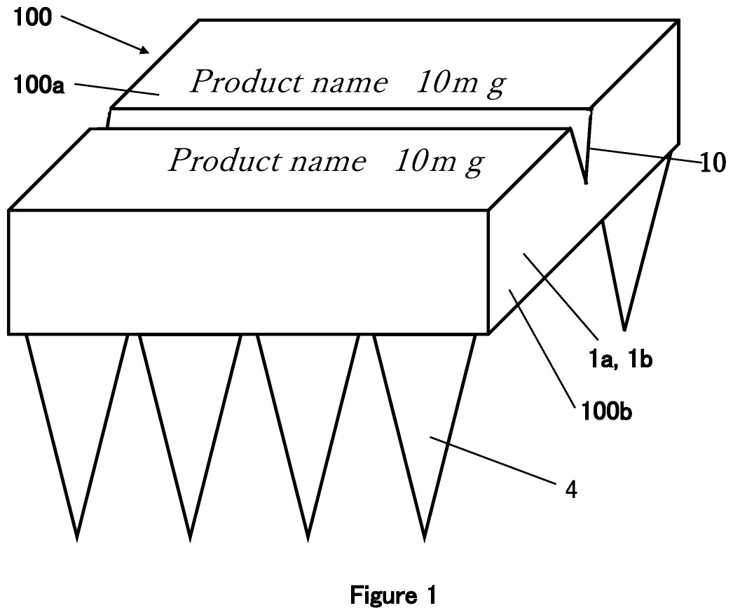

Several examples are depicted below. Possible embodiments of the present invention are not limited to those depicted in FIGS. 1 through 6. For example, the integumental dissolving needle device 100 is depicted as a rectangular solid, but other shapes are possible. FIG. 1 is a perspective view of a groove-type integumental (e.g. skin, scales, bark) dissolving needle device 100, with dosage or dosages printed on the surface or surfaces of each section to make explicit how many milligrams of the ingredient are present per unit area. Groove(s) 10 may be located on the same side of the integumental dissolving needle device 100 as the integumental dissolving needles 4, the opposite side, or both sides. As a representative example, FIG. 1 depicts such an integumental dissolving needle device 100 in which each section contains 10 mg of ingredient, and in which a single groove 10 is located on the side opposite the integumental dissolving needles 4.

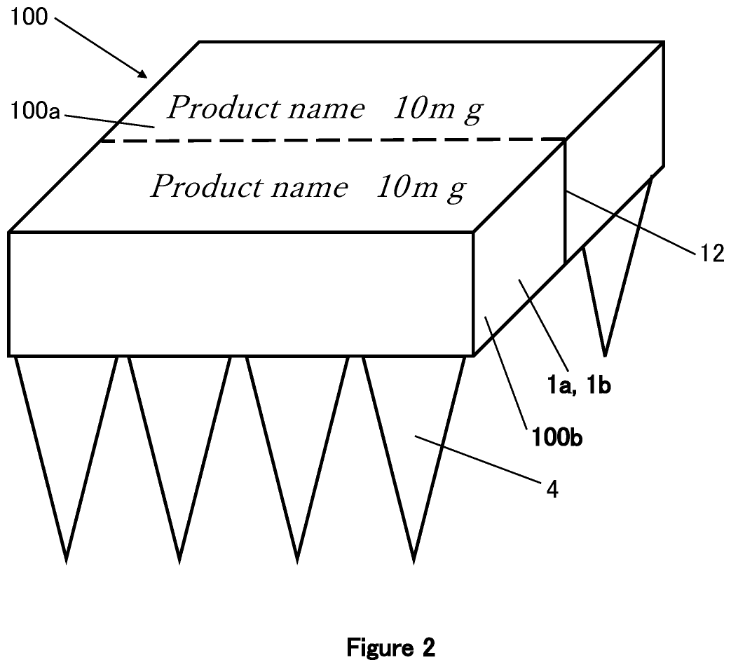

FIG. 2 is a perspective view of a perforation-type integumental (e.g. skin, scales, bark) integumental dissolving needle device 100, with dosage or dosages printed on the surface or surfaces of each section to make explicit how many milligrams of the ingredient are present per unit area. In this example, each section contains 10 mg of ingredient.

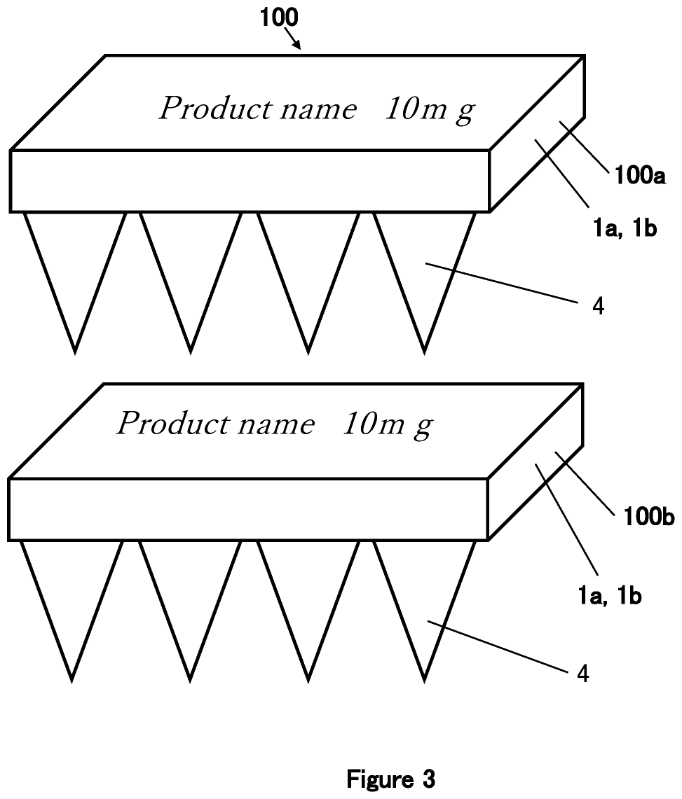

FIG. 3 is a perspective view of a pre-sectioned integumental (e.g. skin, scales, bark) dissolving needle device 100, with dosage or dosages printed on the surface or surfaces of each section 100a, 100b to make explicit how many milligrams of the ingredient are present per unit area. In this example, each section 100a, 100b contains 10 mg of ingredient.

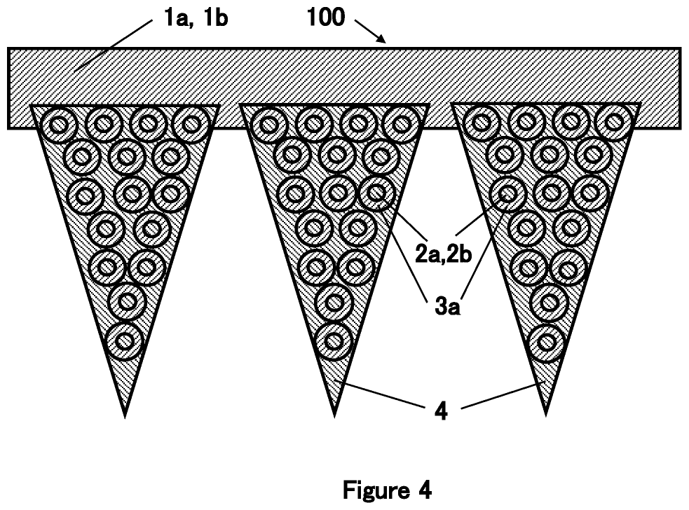

FIG. 4 is a cross-section view of an integumental dissolving needle device 100, in which two-layer granules—consisting of a cosmetic ingredient 2b that is a nanoparticle or that is a micronized particle having a size smaller than a nanometer order (or similarly micronized pharmaceutical ingredient particle 2a), encapsulated by a layer of coating agent 3a that is absorbed into the integument (e.g. skin, scales, bark)—are housed in the integumental (e.g. skin, scales, bark) dissolving needles 4.

FIG. 5 is a cross-section view of an integumental dissolving needle device 100, in which a mixture of two-layer granules and four-layer granules—consisting of a cosmetic ingredient 2b that is a nanoparticle or that is a micronized particle having a size smaller than a nanometer order (or similarly micronized pharmaceutical ingredient particle 2a), encapsulated by a layer of coating agent 3a that is absorbed into the integument (e.g. skin, scales, bark), further coated with an additional layer of micronized cosmetic ingredient 2b (or pharmaceutical ingredient 2a), followed by an additional layer of coating agent 3b—are housed in the integumental (e.g. skin, scales, bark) dissolving needles 4. As a representative example, FIG. 5 depicts some granules as having a four-layer structure; however, granules may have more than four layers.

FIG. 6 is a cross-section view of an integumental dissolving needle device 100, in which four-layer granules—consisting of a cosmetic ingredient 2b that is a nanoparticle or that is a micronized particle having a size smaller than a nanometer order (or similarly micronized pharmaceutical ingredient particle 2a), encapsulated by a layer of coating agent 3a that is absorbed into the integument (e.g. skin, scales, bark), further coated with an additional layer of micronized cosmetic ingredient 2b (or pharmaceutical ingredient 2a), followed by an additional layer of coating agent 3b—are housed in the integumental (e.g. skin, scales, bark) dissolving needles 4. As a representative example, FIG. 5 depicts the granules as having a four-layer structure; however, granules may have more than four layers. Industrial Applicability

The present invention is not exclusively for use for humans: it may be used for animal and plant species as well, giving it high applicability in veterinary medicine and agriculture industries.

### REFERENCE SIGNS LIST

1a poultice
1b patch
2a micronized pharmaceutical ingredient
2b micronized cosmetic ingredient
3a layer of coating agent
3b additional layer of coating agent
4 integumental dissolving needle
10 groove(s)
12 perforation
100 integumental dissolving needle device
100a, 100b sections

What is claimed is:

1. An integumental dissolving needle device for delivery of one or more pharmaceuticals, or one or more cosmetics, into skin, scales, bark, or other integumental tissue, comprising:
a patch;
a dissolving needle provided on the patch;
multi-layer granules housed by the dissolving needle, the multi-layer granules comprising:
a micronized particle of a first pharmaceutical or cosmetic,
a coating agent layer that is to be absorbed into the integument, the coating agent layer encapsulating the micronized particle,
a layer of either the first pharmaceutical or cosmetic, or a second pharmaceutical or cosmetic different from the first pharmaceutical or cosmetic, covering the coating agent layer, and
an outer coating layer that is to be absorbed into the integument, the outer coating layer encapsulating the layer of either the first or second pharmaceutical or cosmetic, wherein the patch does not contain multilayered granules of pharmaceutical or cosmetic ingredients.

2. The integumental dissolving needle device for delivery of one or more pharmaceuticals, or one or more cosmetics, into skin, scales, bark, or other integumental tissue according to claim 1, further comprising:
additional granules housed by the dissolving needle, the additional granules comprising:
a micronized particle of the first pharmaceutical or cosmetic, and
an outer coating layer that is to be absorbed into the integument, the outer coating layer encapsulating the micronized particle.

3. The integumental dissolving needle device for delivery of one or more pharmaceuticals, into skin, scales, bark, or other integumental tissue according to claim 1, further comprising:
additional granules housed by the dissolving needle, the additional granules comprising:
another micronized particle of the pharmaceutical, and
another outer coating layer that is to be absorbed into the integument,
the outer coating layer encapsulating the micronized particle.

4. The integumental dissolving needle device according to claim 1, wherein the dissolving needle itself is composed of one of:
one or more pharmaceuticals;
one or more cosmetics;
one or more pharmaceuticals encapsulated by a coating layer that is absorbed into the integument; and
one or more cosmetics encapsulated by a coating layer that is absorbed into the integument.

5. The integumental dissolving needle device according to claim 1, wherein the dissolving needle itself is composed of one of:
one or more pharmaceuticals; and
one or more pharmaceuticals encapsulated by a coating layer that is absorbed into the integument.

6. The integumental dissolving needle device according to claim 1, wherein the dissolving needle itself is composed of one of:
one or more cosmetics; and
one or more cosmetics encapsulated by a coating layer that is absorbed into the integument.

7. The integumental dissolving needle device according to claim 2, wherein the dissolving needle itself is composed of one of:
one or more pharmaceuticals;
one or more cosmetics;
one or more pharmaceuticals encapsulated by a coating layer that is absorbed into the integument; and
one or more cosmetics encapsulated by a coating layer that is absorbed into the integument.

8. The integumental dissolving needle device according to claim 2, wherein the dissolving needle itself is composed of one of:
one or more pharmaceuticals; and
one or more pharmaceuticals encapsulated by a coating layer that is absorbed into the integument.

9. The integumental dissolving needle device according to claim 2, wherein the dissolving needle itself is composed of one of:
one or more cosmetics; and
one or more cosmetics encapsulated by a coating layer that is absorbed into the integument.

10. A needle device comprising one or more of the integumental dissolving needle device described in claim 1, provided on a surface of a poultice or surfaces of the poultice.

11. A needle device comprising one or more of the integumental dissolving needle device described in claim 2, provided on a surface of a poultice or surfaces of the poultice.

12. A needle device comprising one or more of the integumental dissolving needle device described in claim 4, provided on a surface of a poultice or surfaces of the poultice.

13. The integumental dissolving needle device according to claim 1, wherein:
the integumental dissolving needle device is sectionable, and includes a product name or product names and dosage or dosages written on a surface or surfaces of the integumental dissolving needle device.

14. The integumental dissolving needle device according to claim 2, wherein:
the integumental dissolving needle device is sectionable, and includes a product name or product names and dosage or dosages written on a surface or surfaces of the integumental dissolving needle device.

15. The integumental dissolving needle device according to claim 4, wherein:
the integumental dissolving needle device is sectionable, and includes a product name or product names and dosage or dosages written on a surface or surfaces of the integumental dissolving needle device.

16. The integumental dissolving needle according to claim 1, wherein the multi-layer granules further include a structure in which
(1) the layer of either the first or second pharmaceutical or cosmetic, and
(2) the outer coating layer that is to be absorbed into the integument are all repeated.

17. The integumental dissolving needle according to claim 2, wherein the multi-layer granules further include a structure in which the layer of either the first or second pharmaceutical or cosmetic, and the outer coating layer are repeated.

18. The integumental dissolving needle according to claim 4, wherein the multi-layer granules further include a structure in which the layer of either the first or second pharmaceutical or cosmetic, and the outer coating layer are repeated.

19. The needle device according to claim 10, wherein: the poultice is comprised of a tape or surfaces of the poultice are comprised of tapes.

20. The needle device according to claim 12, wherein: the poultice is comprised of a tape or surfaces of the poultice are comprised of tapes.

21. The integumental dissolving needle device according to claim 1, wherein the coating agent is phosphate ester or phospholipids.

22. The integumental dissolving needle device according to claim 2, wherein the coating agent is phosphate ester or phospholipids.

23. The integumental dissolving needle device according to claim 4, wherein the coating agent is phosphate ester or phospholipids.

24. The needle device according to claim 20, wherein the coating agent is phosphate ester or phospholipids.
